(19) United States
(12) Patent Application Publication
(10) Pub. No.: US 2021/0145917 A1
(43) Pub. Date: May 20, 2021

(54) Composition having effect of alleviating bromhidrosis

(71) Applicant: Ajou Medics Co.,Ltd, Seongnam-si (KR)

(72) Inventor: SUNG NACK LEE, Seongnam-si (KR)

(21) Appl. No.: 16/685,492

(22) Filed: Nov. 15, 2019

(51) Int. Cl. A61K 36/78; A61K 31/045; A61K 47/10; A61K 31/4166; A61K 31/704; A61K 31/125; A61K 36/53; A61K 31/60; A61K 31/14; A61P 43/00

(52) U.S. Cl. CPC A61K 36/78; A61K 31/045; A61K 47/10; A61K 31/4166; A61P 43/00; A61K 31/125; A61K 36/53; A61K 31/60; A61K 31/14; A61K 31/704

(57) ABSTRACT

Disclosed is a composition having an effect of alleviating bromhidrosis which contains dipotassium glycyrrhizinate, benzalkonium chloride, DL-camphor, a Houttuynia cordata extract and the like in order to eliminate side effects associated with triclosan. Further, disclosed is a composition having an effect of alleviating symptoms of bromhidrosis containing 11.5 parts by weight of purified water, 0.5 parts by weight of salicylic acid, 0.2 parts by weight of a fragrance, 7.5 parts by weight of butylene glycol, 0.055 parts by weight of allantoin, 0.255 parts by weight of dipotassium glycyrrhizinate, 0.03 parts by weight of benzalkonium chloride, 0.055 parts by weight of DL-camphor, 0.505 parts by weight of lavender oil and 0.505 parts by weight of a Houttuynia cordata extract, with reference to 80 parts by weight of ethanol.

### BACKGROUND OF THE INVENTION

### Field of the Invention

Applicant states that the subject matter of this invention was disclosed in Korea Patent Application Publication No. 10-2018-0130074, published Dec. 6, 2018, which is not considered as prior art under 35 USC 102(b)(1) as exception.

The present invention relates to a composition having an effect of alleviating bromhidrosis (sweat odor), and more particularly to a composition having an effect of alleviating bromhidrosis which contains dipotassium glycyrrhizinate, benzalkonium chloride, DL-camphor, a Houttuynia cordata extract and the like in order to eliminate side effects associated with triclosan.

### Description of the Related Art

More than 2 million sweat glands are distributed throughout the human skin, and most sweat is secreted from apocrine sweat glands and is a colorless and odorless clear liquid that contains some salt.

Meanwhile, sweat and the cells that are attached thereto are isolated from apocrine sweat glands, which are distributed in the armpits, breasts, pubis and anus, and undergo decapitation secretion. The protein component of the secreted cellular parts is degraded by bacteria in the armpits, emitting an unpleasant odor as a result of biological processes, that is, decay.

Generally, there are passive methods of frequently washing the body or using a spray containing fragrance, called “deodorant”, but these methods have not been very satisfactory in terms of its effect to date.

In recent years, apocrine sweat glands concentrated in the armpit area have been removed by a surgical operation using a laser. However, the surgical operation is not only economically burdensome, it also limits social activities that may occur after the procedure, thus being time-consuming. In particular, apocrine glands are distributed not only in the armpits, but also in the breasts and pubic area, so surgical treatment thereof is limited.

Bromhidrosis refers to a phenomenon in which an irritating and unpleasant odor is emitted from the armpit, which is mainly caused by a substance secreted from the apocrine glands distributed in the armpit and begins after puberty when the secretory function becomes active. Odors restrict young women in particular from social activities. Bromhidrosis may be inhibited by reducing the number of bacteria and suppressing secretion from the apocrine glands.

Most people wash with antibacterial soap, apply antibiotics to the skin, or use perfumes, or cosmetics that reduce sweat secretion. In Korea, even though bromhidrosis is not severe, people tend to be sensitive to odors. Therefore, non-surgical treatment is generally used rather than surgical treatment.

However, conventionally used formulations have not had a great effect on alleviating bromhidrosis symptoms.

In order to solve the above problems, the present applicant filed a patent application on Jul. 16, 2003, as Korean Patent Application No. 10-2003-0048890 (Title of the invention: Composition having effect of alleviating symptoms of bromhidrosis), and claimed a “composition having effect of alleviating symptoms of bromhidrosis containing 20.0 to 80.0 parts by weight of ethanol, 5.0 to 10.0 parts by weight of polyhydric alcohol, 0.01 to 0.2 parts by weight of glycyrrhizic acid (licorice acid) or a dialkali metal salt thereof, 0.1 to 0.5 parts by weight of salicylic acid, 0.1 to 0.5 parts by weight of triclosan, 0.01 to 0.1 parts by weight of a fragrance essence, 0.1 to 0.3 parts by weight of allantoin, 0.01 to 0.1 parts by weight of DL-camphor, 0.01 to 1.0 parts by weight of a Houttuynia cordata extract, 0.01 to 1.0 parts by weight of a green tea extract, 0.01 to 1.0 parts by weight of an aloe extract and 5.0 to 30.0 parts by weight of water.”

However, the composition having the effect of alleviating bromhidrosis symptoms has a problem in that side effects associated with triclosan may occur.

### SUMMARY OF THE INVENTION

Therefore, the present invention has been made in view of the above problems, and it is one object of the present invention to provide a composition having the effect of alleviating bromhidrosis symptoms to eliminate side effects associated with triclosan.

However, the object of the present invention is not limited to those described above, and other objects not mentioned herein will be clearly understood from the following description by those skilled in the art.

In accordance with the present invention, the above and other objects can be accomplished by the provision of a composition having the effect of alleviating symptoms of bromhidrosis containing 11.5 parts by weight of purified water, 0.5 parts by weight of salicylic acid, 0.2 parts by weight of a fragrance, 7.5 parts by weight of butylene glycol, 0.055 parts by weight of allantoin, 0.255 parts by weight of dipotassium glycyrrhizinate, 0.03 parts by weight of benzalkonium chloride, 0.055 parts by weight of DL-camphor, 0.505 parts by weight of lavender oil and 0.505 parts by weight of a Houttuynia cordata extract, with reference to 80 parts by weight of ethanol.

### BRIEF DESCRIPTION OF THE DRAWINGS

FIG. 1 is an image showing results of tests on the armpit area with Acinetobacter calcoaceticus (KCTC 2334) using a general antibiotic;

FIG. 2 is an image showing results of tests on the armpit area with Corynebacterium minutissimum (ATCC 23346) using a general antibiotic; and

FIG. 3 is an image showing results of tests on the armpit area using the compositions having the effect of alleviating symptoms of bromhidrosis according to the present invention.

### DETAILED DESCRIPTION OF THE INVENTION

Hereinafter, preferred embodiments of the composition having the effect of alleviating symptoms of bromhidrosis will be described in detail. In the following description of the present invention, detailed descriptions of known functions and configurations incorporated herein will be omitted when the same may obscure the subject matter of the present invention.

The composition having the effect of alleviating symptoms of bromhidrosis contains ethanol, purified water, salicylic acid, a fragrance, butylene glycol, allantoin, dipotassium glycyrrhizinate, benzalkonium chloride, DL-camphor, lavender oil and a Houttuynia cordata extract.

The composition having the effect of alleviating symptoms of bromhidrosis according to the present invention will be described in detail.

Ethanol is also called “ethyl alcohol” and is the alcohol that is the main ingredient of alcoholic spirits. It has the molecular formula C₂H₆O. Chain hydrocarbons are also called aliphatic hydrocarbons and their names vary depending on the number of carbons.

Also, ethanol is an aliphatic hydrocarbon derivative in which a hydrogen atom is substituted with a hydroxy group (—OH) in ethane (C₂H₆) having two carbon atoms in the aliphatic hydrocarbon compound. Ethanol is a colorless liquid emitting a unique scent, has a melting point of −114° C. and a boiling point of 78° C., and is anesthetic.

In addition, ethanol is used as a synthetic beverage, a solvent, a fuel, an antifreeze agent and an intermediate of organic synthesis, and is also used as a disinfectant at high concentrations.

Thus, ethanol is obtained in an amount of 75 to 85 parts by weight.

In addition, purified water is obtained in an amount of 10 to 13 parts by weight with respect to 80 parts by weight of ethanol.

In addition, since salicylic acid is both acidic and phenolic, the salicylic acid becomes purple when an aqueous solution of iron (III) chloride is added thereto.

Salicylic acid is contained in the form of ester in most essential oils. In particular, fragrance oil from leaves of the genus Gaultheria, which is a member of Ericaceae, is produced by reacting dry sodium phenoxide with carbon dioxide while heating and pressurizing, because it contains methyl salicylate as a main ingredient.

In addition, the salicylic acid has antipyretic and analgesic activities and is thus administered as a sodium or calcium salt, and binds to various drugs and is thus used to improve solubility, stability and pharmacological activity.

In addition, salicylic acid is used as a plaster to remove pathological proliferation (warts and corns) of keratin, methyl ester is used as an external coating agent for analgesic and anti-inflammatory application, and phenyl ester is used as an antiseptic for the intestines, urethra or the like.

Thus, salicylic acid is obtained in an amount of 0.3 to 0.7 parts by weight with respect to 80 parts by weight of ethanol.

In addition, the fragrance is a substance emitting a fragrance.

Thus, the fragrance of one selected from orange, lemon, anisole, eugenol, 1-carbon, coumarin, geraniol, citral, citronella, vanillin, nonalactone, borneol, maltol, menthol, limonene, chamomile, clary sage, bergamot, mandarin, rosemary and laurel tree is obtained at 0.1 to 0.3 parts by weight with reference to 80 parts by weight of ethanol.

Also, butylene glycol is mainly used as a solvent for cosmetics, and serves to dissolve cosmetic ingredients. Butylene glycol attracts moisture and is thus used as a moisturizer. Butylene glycol is mainly used as a moisturizer, skin conditioner, solvent, viscosity-reducing agent or fragrance.

Therefore, butylene glycol is obtained in an amount of 5 to 10 parts by weight with respect to 80 parts by weight of ethanol.

In addition, allantoin is a natural substance extracted mainly from shoots of perennial plants, for example, comfrey, oak, wheat and the like. Allantoin is obtained by purifying urate oxide produced by catabolism, which is a process of degrading high-molecular-weight organic substances into low-molecular-weight substances.

Allantoin increases the moisture content of interstitial cells between living and dead cells to remove keratin, moisturizes the skin to make the skin soft, and promotes the germination of cells to help heal wounds of the skin. Allantoin also has the effect of protecting skin that is sensitive to irritation and antigens. Allantoin is used as a therapeutic agent rather than steroid therapy for the treatment of pneumonia or atopic dermatitis. In addition, allantoin is used as a component of medicines or cosmetics to promote metabolism through biochemical reprocessing. In addition, no cases of allergies or side effects caused by allantoin have been reported, and allantoin is thus used as a component of cosmetics. Allantoin is known to be effective in protecting the skin and is much safer than other chemicals.

Allantoin is mainly used in oral hygiene products such as toothpaste and mouthwash, as well as in cosmetics such as shampoo, acne medicine, sunburn relief cream and cosmetic cream and medicines.

Thus, allantoin is obtained from camphor in an amount of 0.01 to 0.1 parts by weight with respect to 80 parts by weight of ethanol.

In addition, dipotassium glycyrrhizinate (also referred to as “glycyrrhizic acid or alkali metal salt thereof”) is obtained from Glycyrrhiza glabra (licorice), which is a medicinal plant. Licorice has reddish-brown roots that grow deep into the ground and angular stems, and grows straight to about 1 m. In addition, since white hairs of licorice grow thick, licorice appear light gray with glandular dots scattered therein. The alternating leaves are imparipinnate compound leaves. Licorice has 7 to 17 small leaves which are egg-shaped with pointed ends. Small leaves have a length of 2 to 5 cm and a width of 1 to 3 cm and include white hairs and glandular dots on both sides thereof with no sawtooth edge.

Licorice blooms in July to August, and has purple flowers 1.4 to 2.5 cm long, borne in the form of a raceme on the axil. The pod is thin and long like a line and is bent like a bow and contains 6 to 8 kidney-shaped seeds. The root tastes sweet and is thus used as a sweetener and herbal medicine.

Thus, dipotassium glycyrrhizinate is obtained from licorice (Glycyrrhiza glabra) in an amount of 0.01 to 0.5 parts by weight with respect to 80 parts by weight of ethanol.

Benzalkonium chloride is a mixture component of alkyl benzyl dimethylammonium chloride.

Benzalkonium chloride has an antibacterial effect.

Thus, the benzalkonium chloride is obtained in an amount of 0.01 to 0.05 parts by weight with respect to 80 parts by weight of ethanol.

In addition, DL-camphor acts on the skin sensation and provides a cool feeling.

Thus, DL-camphor is obtained in an amount of 0.01 to 0.1 parts by weight with respect to 80 parts by weight of ethanol.

Lavender blooms light purple or white from June to September and the flowers are borne sparse in the form of a spike at the end of a long peduncle without leaves. There is an oil fountain emitting a scent between the hairs that cover the flowers, leaves and stems.

Lavender is used as a raw material for perfumes and cosmetics and as a fragrance for cooking, as well as to treat headaches and nervousness.

Thus, lavender oil is obtained from lavender in an amount of 0.01 to 1 parts by weight with respect to 80 parts by weight of ethanol.

In addition, fish mint is a perennial herb belonging to the Saururaceae family, the scientific name is Houttuynia cordata, and the extract of this herb is a fish mint (Houttuynia cordata) extract. This extract is known to have detoxification, intestinal regulation, blood-clearing and antibacterial activities.

Thus, a Houttuynia cordata extract is obtained from Houttuynia cordata in an amount of 0.01 to 1 parts by weight with respect to 80 parts by weight of ethanol.

In order to determine the effect of alleviating bromhidrosis depending on the content of the ingredients contained in the composition, three different compositions were prepared and applied to 10 clinical subjects. After 12 hours, a test to identify whether or not bromhidrosis was alleviated was performed. The embodiments of three compositions used herein are as follows.

Hereinafter, a first embodiment of a method for preparing the composition having an effect of alleviating bromhidrosis according to the present invention will be described in brief.

First, 80 parts by weight of ethanol is obtained and then 10 parts by weight of purified water is obtained with respect to 80 parts by weight of ethanol.

Also, salicylic acid is obtained in an amount of 0.3 parts by weight with respect to 80 parts by weight of ethanol.

Also, a fragrance is obtained in an amount of 0.1 parts by weight with respect to 80 parts by weight of ethanol.

Also, butylene glycol is obtained in an amount of 5 parts by weight with respect to 80 parts by weight of ethanol.

Also, after obtaining comfrey, allantoin is obtained from the comfrey in an amount of 0.01 parts by weight with respect to 80 parts by weight of ethanol.

Also, after obtaining Glycyrrhiza glabra (licorice), dipotassium glycyrrhizinate is obtained from the Glycyrrhiza glabra (licorice) in an amount of 0.01 parts by weight with respect to 80 parts by weight of ethanol.

Also, benzalkonium chloride having an antimicrobial effect is obtained in an amount of 0.01 parts by weight with respect to 80 parts by weight of ethanol.

Also, DL-camphor, which acts on the skin sensation and provides a cool feeling, is obtained in an amount of 0.01 parts by weight with respect to 80 parts by weight of ethanol.

Also, after obtaining lavender, lavender oil is obtained from the lavender in an amount of 0.01 parts by weight with respect to 80 parts by weight of ethanol.

Also, after obtaining Houttuynia cordata, a Houttuynia cordata extract is obtained from the Houttuynia cordata in an amount of 0.01 parts by weight with respect to 80 parts by weight of ethanol.

Also, 80 parts by weight of ethanol, 10 parts by weight of purified water, 0.3 parts by weight of salicylic acid, 0.1 parts by weight of a fragrance, 5 parts by weight of butylene glycol, 0.01 parts by weight of allantoin, 0.01 parts by weight of dipotassium glycyrrhizinate, 0.01 parts by weight of benzalkonium chloride, 0.01 parts by weight of DL-camphor, 0.01 parts by weight of lavender oil and 0.01 parts by weight of a Houttuynia cordata extract, obtained as noted above, are mixed together.

Hereinafter, a second embodiment of a method for preparing the composition having an effect of alleviating bromhidrosis according to the present invention will be described in brief.

First, 80 parts by weight of ethanol is obtained, and then 11.5 parts by weight of purified water is obtained with respect to 80 parts by weight of ethanol.

Also, salicylic acid is obtained in an amount of 0.5 parts by weight with respect to 80 parts by weight of ethanol.

Also, a fragrance is obtained in an amount of 0.2 parts by weight with respect to 80 parts by weight of ethanol.

Also, butylene glycol is obtained in an amount of 7.5 parts by weight with respect to 80 parts by weight of ethanol.

Also, after obtaining comfrey, allantoin is obtained from the comfrey in an amount of 0.055 parts by weight with respect to 80 parts by weight of ethanol.

Also, after obtaining Glycyrrhiza glabra (licorice), dipotassium glycyrrhizinate is obtained from the Glycyrrhiza glabra (licorice) in an amount of 0.255 parts by weight with respect to 80 parts by weight of ethanol.

Also, benzalkonium chloride having an antimicrobial effect is obtained in an amount of 0.03 parts by weight with respect to 80 parts by weight of ethanol.

Also, DL-camphor, which acts on the skin sensation and provides a cool feeling, is obtained in an amount of 0.055 parts by weight with respect to 80 parts by weight of ethanol.

Also, after obtaining lavender, lavender oil is obtained from the lavender in an amount of 0.505 parts by weight with respect to 80 parts by weight of ethanol.

Also, after obtaining Houttuynia cordata, a Houttuynia cordata extract is obtained from the Houttuynia cordata in an amount of 0.505 parts by weight with respect to 80 parts by weight of ethanol.

Also, 80 parts by weight of ethanol, 11.5 parts by weight of purified water, 0.5 parts by weight of salicylic acid, 0.2 parts by weight of a fragrance, 7.5 parts by weight of butylene glycol, 0.055 parts by weight of allantoin, 0.255 parts by weight of dipotassium glycyrrhizinate, 0.03 parts by weight of benzalkonium chloride, 0.055 parts by weight of DL-camphor, 0.505 parts by weight of lavender oil and 0.505 parts by weight of a Houttuynia cordata extract, obtained as noted above, are mixed together.

Hereinafter, a third embodiment of a method for preparing the composition having an effect of alleviating bromhidrosis according to the present invention will be described in brief.

First, 80 parts by weight of ethanol is obtained and then 13 parts by weight of purified water is obtained with respect to 80 parts by weight of ethanol.

Also, salicylic acid is obtained in an amount of 0.7 parts by weight with respect to 80 parts by weight of ethanol.

Also, a fragrance is obtained in an amount of 0.3 parts by weight with respect to 80 parts by weight of ethanol.

Also, butylene glycol is obtained in an amount of 10 parts by weight with respect to 80 parts by weight of ethanol.

Also, after obtaining comfrey, allantoin is obtained from the comfrey in an amount of 0.1 parts by weight with respect to 80 parts by weight of ethanol.

Also, after obtaining Glycyrrhiza glabra (licorice), dipotassium glycyrrhizinate is obtained from the Glycyrrhiza glabra (licorice) in an amount of 0.5 parts by weight with respect to 80 parts by weight of ethanol.

Also, benzalkonium chloride having an antimicrobial effect is obtained in an amount of 0.05 parts by weight with respect to 80 parts by weight of ethanol.

Also, DL-camphor, which acts on the skin sensation and provides a cool feeling, is obtained in an amount of 0.1 parts by weight with respect to 80 parts by weight of ethanol.

Also, after obtaining lavender, lavender oil is obtained from the lavender in an amount of 1 part by weight with respect to 80 parts by weight of ethanol.

Also, after obtaining Houttuynia cordata, a Houttuynia cordata extract is obtained from the Houttuynia cordata in an amount of 1 part by weight with respect to 80 parts by weight of ethanol.

Also, 80 parts by weight of ethanol, 13 parts by weight of purified water, 0.7 parts by weight of salicylic acid, 0.3 parts by weight of a fragrance, 10 parts by weight of butylene glycol, 0.1 parts by weight of allantoin, 0.5 parts by weight of dipotassium glycyrrhizinate, 0.05 parts by weight of benzalkonium chloride, 0.1 parts by weight of DL-camphor, 1 part by weight of lavender oil and 1 part by weight of a Houttuynia cordata extract, obtained as noted above, are mixed together.

The compositions of Example 1, Example 2 and Example 3 were applied to the axillary area of 10 clinical subjects suffering from bromhidrosis, and after 12 hours, the diagnosis results were obtained.

TABLE 1

| | Composition | Composition | Composition |
|---|---|---|---|
| | of first | of second | of third |
| | embodiment | embodiment | embodiment |
| Excellent effect of | 4 | 7 | 3 |
| alleviating bromhidrosis | | | |
| Slight effect of | 5 | 3 | 5 |
| alleviating bromhidrosis | | | |
| No effect of | 1 | 0 | 2 |
| alleviating bromhidrosis | | | |

Therefore, as shown in Table 1, all three compositions are shown to be effective in alleviating bromhidrosis without side effects associated with triclosan, and in particular, the composition of the second embodiment is effective in quickly alleviating bromhidrosis symptoms.

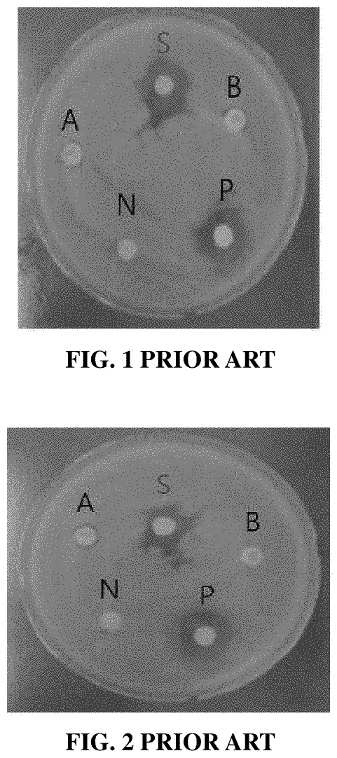

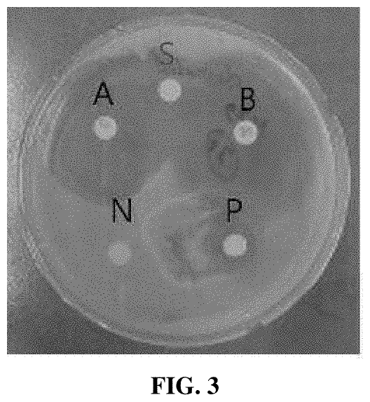

FIGS. 1 and 2 are images showing the results of tests on the armpit area using a general antibiotic, and FIG. 3 is an image showing the results of tests on the armpit area using the compositions having the effect of alleviating symptoms of bromhidrosis according to the present invention.

Referring to FIGS. 1-3, the following tests were conducted to objectively verify the antimicrobial activity of the composition against the three types of flora in the armpit area which cause bromhidrosis.

Implementer: Department of Cell Biology, Ajou University School of Medicine

Purpose of test: the three types of flora always present in the armpit area, i.e., Acinetobacter calcoaceticus (KCTC 2334), Corynebacterium minutissimum (ATCC 23346), and Brevibacterium sp. (KCTC 1679) degrade the proteins contained in sweat secreted from the armpit area and thus cause an unpleasant odor. Therefore, the antimicrobial activities against the three types of bacteria are measured to determine whether or not the bacteria that cause the odor can be removed.

P: Positive control: Clindamycin Phosphate 1% external preparation (Product Name: DuoClean Liquid)

N: Negative control: purified water

S: Composition having effect of alleviating bromhidrosis symptoms according to the present invention

A: AMPICILLIN/B: BACITRACIN (generic antibiotics)

The test results showed that the composition according to the present invention has antimicrobial activity similar to raw materials of antibiotics used in medicine and excellent antibacterial activity against axillary flora, which cause odors, and thus is expected to be effective in preventing odors.

As apparent from the foregoing, the composition having an effect of alleviating bromhidrosis according to the present invention contains dipotassium glycyrrhizinate, benzalkonium chloride, DL-camphor, a Houttuynia cordata extract and the like, thereby having an effect of eliminating side effects associated with triclosan.

As a result, the composition according to the present invention has the effect of alleviating bromhidrosis symptoms.

The detailed description of the present invention is merely exemplary, is given only to illustrate the present invention and is not intended to limit the meaning or the scope of the invention as defined in claims. Therefore, those skilled in the art will understand that various modifications and other embodiments equivalent thereto are possible. Therefore, the intended scope of the technical protection of the present invention is to be defined by the technical concept of the appended claims.

What is claimed is:

1. A composition having an effect of alleviating symptoms of bromhidrosis comprising:
ethanol;
purified water;
salicylic acid;
a fragrance;
butylene glycol;
allantoin;
dipotassium glycyrrhizinate;
benzalkonium chloride;
DL-camphor;
lavender oil; and
a Houttuynia cordata extract.

2. A composition having an effect of alleviating symptoms of bromhidrosis comprising:
10 to 13 parts by weight of purified water;
0.3 to 0.7 parts by weight of salicylic acid;
0.1 to 0.3 parts by weight of a fragrance;
5 to 10 parts by weight of butylene glycol;
0.01 to 0.1 parts by weight of allantoin;
0.01 to 0.5 parts by weight of dipotassium glycyrrhizinate;
0.01 to 0.05 parts by weight of benzalkonium chloride;
0.01 to 0.1 parts by weight of DL-camphor;
0.01 to 1 parts by weight of lavender oil; and
0.01 to 1 parts by weight of a Houttuynia cordata extract, with reference to 80 parts by weight of ethanol.

3. A composition having an effect of alleviating symptoms of bromhidrosis comprising:
11.5 parts by weight of purified water;
0.5 parts by weight of salicylic acid;
0.2 parts by weight of a fragrance;
7.5 parts by weight of butylene glycol;
0.055 parts by weight of allantoin;
0.255 parts by weight of dipotassium glycyrrhizinate;
0.03 parts by weight of benzalkonium chloride;
0.055 parts by weight of DL-camphor;
0.505 parts by weight of lavender oil; and
0.505 parts by weight of a Houttuynia cordata extract, with reference to 80 parts by weight of ethanol.
